(19) United States
(12) Patent Application Publication
(10) Pub. No.: US 2024/0389927 A1
(43) Pub. Date: Nov. 28, 2024

(54) USE OF GASTRIC EMPTYING TO PREDICT AND MANAGE GLYCEMIA IN DIABETES MELLITUS

(71) Applicant: Mayo Foundation for Medical Education and Research, Rochester, MN (US)

(72) Inventors: Adil E. Bharucha, Rochester, MN (US); Yogish C. Kudva, Rochester, MN (US); Armando Manduca, Rochester, MN (US)

(73) Assignee: Mayo Foundation for Medical Education and Research, Rochester, MN (US)

(21) Appl. No.: 18/671,347

(22) Filed: May 22, 2024

Related U.S. Application Data
(60) Provisional application No. 63/468,948, filed on May 25, 2023.

(51) Int. Cl. A61B 5/00; A61B 5/083

(52) U.S. Cl. CPC A61B 5/4238; A61B 5/083; A61B 5/4839

(57) ABSTRACT

This document describes systems and methods for managing glycemia in patients with diabetes mellitus by optimizing insulin delivery dosages and timing. For example, this document describes systems and methods that utilize a patient's gastric emptying rate and other parameters to determine the optimal insulin delivery dosages and timing for the patient.

### CROSS-REFERENCE TO RELATED APPLICATIONS

This application claims the benefit of U.S. Patent Application Ser. No. 63/468,948, filed on May 25, 2023. The disclosure of the prior application is considered part of (and is incorporated by reference in) the disclosure of this application.

### BACKGROUND

### 1. Technical Field

This document relates to systems and methods for managing glycemia in patients with diabetes mellitus by optimizing insulin delivery dosages and timing. For example, this document relates to systems and methods that utilize a patient's gastric emptying rate and other parameters to determine the optimal insulin delivery dosages and timing for the patient.

### 2. Background Information

Approximately 50% of patients with type 1 diabetes mellitus (TID) have delayed or rapid gastric emptying (GE). GE can be measured with scintigraphy or the 13C-Spirulina gastric emptying breath test (GEBT), which is an extensively validated, FDA-approved, and office-based test that does not entail radiation. In diabetes mellitus (DM), abnormal GE is often unrecognized because most patients have limited GI symptoms and GE is not routinely measured.

Abnormal GE affects the rate of nutrient absorption. Glucagon-like peptide-1 (GLP1) delays GE and improves glycemic control in type 2 DM (T2D). Acceleration of GE increases postprandial hyperglycemia in T1D and T2D. However, the effects of innate (i.e., not pharmacologically mediated) delayed GE on glycemia in T1D have not been adequately evaluated. Moreover, GE is not used to guide therapy. For example, the dose and timing of insulin delivered either by patients or through pumps in DM do not take into account the patient's GE.

### SUMMARY

This document describes systems and methods for managing glycemia in patients with diabetes mellitus by optimizing insulin delivery dosages and timing. For example, this document describes systems and methods that utilize a patient's gastric emptying rate and other parameters to determine the optimal insulin delivery dosages and timing for the patient.

The systems and methods disclosed herein utilize GE to optimize insulin delivery (i.e., dose and timing) with or without artificial pancreas (AP) automated insulin delivery systems and thereby improve glycemic control in DM. Existing models for deconvoluting glucose variability in DM do not incorporate terms for the rate of GE. Hence, the inventors developed a new model of oral glucose absorption, with terms for GE, and incorporated a sub model of insulin diffusion to predict postprandial continuous glucose monitoring (CGM) glucose levels in TID. The inventors found that subcutaneous glucose values, measured with CGM sensors, for four (4) hours after a meal can be accurately predicted using the new mathematical model that incorporates fasting glucose, ingested calories, insulin delivery, and GE measured with scintigraphy or a GEBT. The predicted postprandial glucose values approximate closely to actual postprandial CGM glucose values only if the equations use the actual GE values for that particular patient.

The inventive work performed by the inventors establishes that GE, evaluated with scintigraphy or a GEBT, is essential for accurately predicting postprandial glycemia in TID. The inventive features described herein can also be advantageously incorporated in insulin hybrid closed loop systems, artificial pancreas (AP) automated insulin delivery systems, or hybrid AID systems.

In one aspect, this disclosure is directed to a method of controlling blood glucose levels of a patient. The method includes delivering one or more dosage amounts of insulin to the patient. The one or more dosage amounts are determined based on a combination of factors specific to the patient that include: (i) a fasting glucose level, (ii) ingested calories, and (iii) a gastric emptying value.

Particular embodiments of the subject matter described in this document can be implemented to realize one or more of the following advantages. In some embodiments, the post-prandial glucose profile can be predicted accurately and precisely from the GE rate in patients with type 1 diabetes. Moreover, the findings of the studies described herein support consideration of assessing GE in patients with suboptimal glycemic control, even in patients with no or mild gastrointestinal symptoms.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIGS. 1 and 2 are graphs that show comparisons between predicted and actual glucose concentrations.

FIG. 3 is a graph that shows the closeness of fit between the actual scintigrahic GE and estimated scintigraphic GE derived from GEBT.

FIG. 4 is a graph that shows a comparison of actual CGM glucose levels and levels predicted based on scintigraphy and GEBT.

FIG. 5 shows representative images comparing predicted and actual CGM in individual patients with rapid, normal, and delayed GE. The rate of increase of glucose is highest in rapid GE, followed by normal and delayed GE.

FIG. 6 shows the reproducibility between two GE studies in ten patients.

FIG. 7 shows the root mean squared error between the actual and predicted glucose in the first and second studies. Filled and open symbols represent patients on MDI (multiple daily injections) or an insulin pump (continuous subcutaneous insulin infusion, CSII).

FIGS. 8 and 9 show the mean percent error between actual and predicted CGM, averaged across patients, at each time point for studies 1 and 2. In the first hour, the errors are negative (i.e., predicted CGM>actual CGM). At all timepoints, these errors are ≤20%.

FIGS. 10 and 11 show individual patient data and mean (SD) for root-mean square error, percent difference between the area under the actual and predicted CGM curve for studies 1 and 2.

FIG. 12 shows the average postprandial 4 hr CGM glucose profile after adjusting the time of prandial insulin administration in patients with low, moderate, and high insulin sensitivity and normal, rapid, or delayed GE. The x-axis shows the time of prandial insulin administration, 0 being concurrent with meal, negative values: before the meal and positive values: after the meal at 10-minute intervals. The y-axis shows the fasting blood glucose, and the color of the heat map reflects the average postprandial 4 hr CGM glucose ranging up to 300 mg/dl. Hypoglycemia is common with 1) higher insulin sensitivity 2) delayed GE. Conversely, hyperglycemia is common in patients with 1) lower insulin sensitivity 2) rapid GE.

FIG. 13 shows the parameter estimates derived from the linear regression model fitted to each of the nine combinations of insulin sensitivity and GE.

Like reference numbers represent corresponding parts throughout.

### DETAILED DESCRIPTION

This document describes systems and methods for managing glycemia in patients with diabetes mellitus by optimizing insulin delivery dosages and timing. For example, this document describes systems and methods that utilize a patient's gastric emptying rate and other parameters to determine the optimal insulin delivery dosages and timing for the patient.

Mathematical models have proven to be useful for understanding postprandial glucose metabolism. These models (e.g., the oral minimal model method) conventionally incorporate measured plasma glucose, insulin, and C-peptide to estimate metabolic parameters (i.e., insulin sensitivity, β-cell responsiveness, and hepatic insulin extraction) in healthy people, and in patients with T2DM or T1DM.

The inventor's work reveals that subcutaneous glucose values (e.g., measured with CGM sensors) for four (4) hours after a meal can be more accurately predicted with a mathematical model that also incorporates the patient's particular GE, measured with scintigraphy or the GEBT (along with the patient's fasting glucose, ingested calories, and insulin delivery). Moreover, the predicted postprandial glucose values approximate closely to actual postprandial CGM glucose values only if the model uses the actual GE values for that patient. This strongly suggests that GE is beneficial, or even required, to predict accurately a patient's postprandial glucose values.

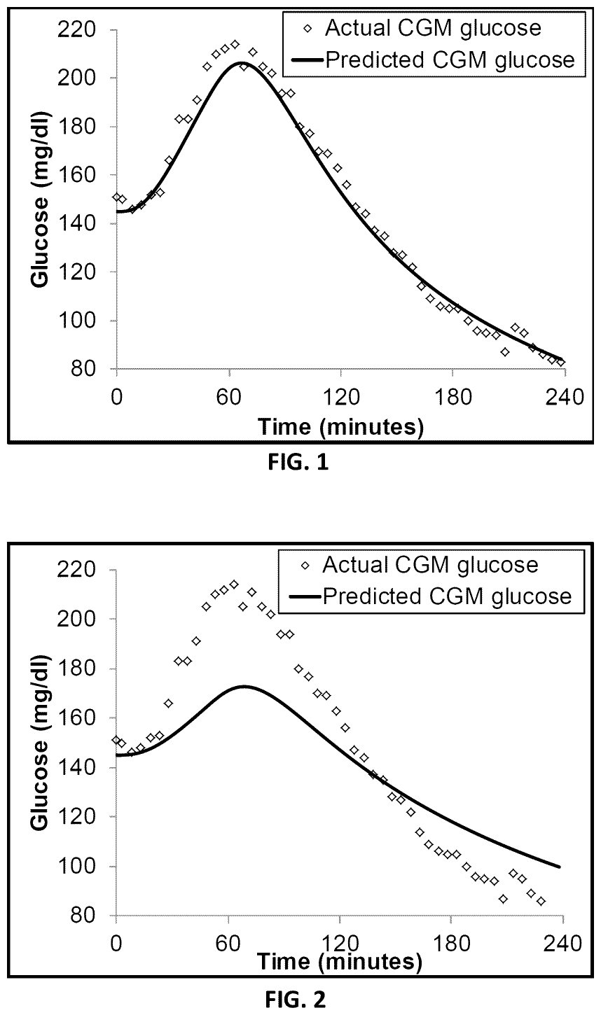

FIGS. 1 and 2 show comparisons between predicted and actual glucose concentrations. In particular, these graphs demonstrate the actual and predicted CGM-glucose concentrations over four (4) hours after a 99 mTc labeled meal (296 kcal, 32% protein, 35% fat, and 33% carbohydrate) in a T1D patient with rapid GE. FIG. 1 shows the results of a model that takes into account the patient's particular GE. FIG. 2 shows the results of a model that does not take into account the patient's particular GE. By comparing these two graphs, it can be seen that taking into account the patient's GE results in a prediction of the patient's postprandial glucose values that is significantly more accurate than when GE is not used for the prediction.

Values for glucose effectiveness at zero insulin (p1), active insulin clearance (p2), insulin-dependent glucose uptake (p3), and the rate of glucose absorption from the small intestine into the plasma (kabs) were fitted in the model with nonlinear least squares, minimizing the sum squared error between actual and predicted CGM values. With the measured GE values for the patient (FIG. 1), the predicted CGM-glucose closely approximated the actual CGM glucose measurements.

For the patient shown, with rapid GE, the root mean square (RMS) error was merely 5.48 mg/dl. The area under the CGM curve from t=0 to t=120 min (AUC), which is an example of a quantity that would be considered clinically relevant, was 21,845 mg-min/dl for the actual measurements and 21,253 mg-min/dl for the predicted (i.e., (2.7% lower) (FIG. 1). When the actual GE value was substituted with a typical value for normal GE, the goodness of fit between predicted and actual CGM values was poorer (FIG. 2); the actual RMS error was 21.4 mg/dl, and the AUC was 19,098 mg-min/dl (12.6% lower).

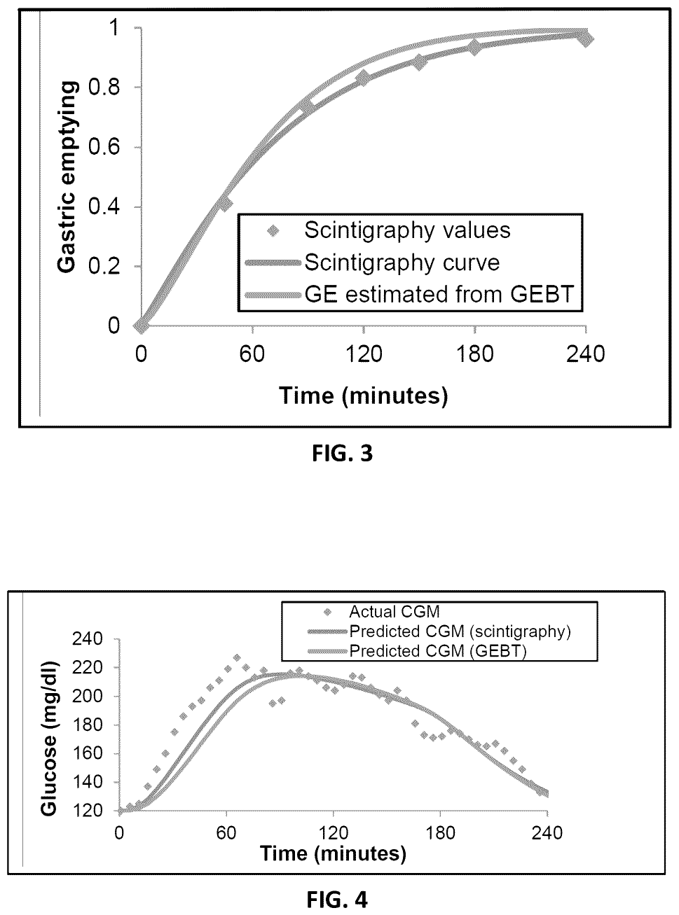

FIG. 3 shows the closeness of fit between the actual scintigrahic GE and estimated scintigraphic GE derived from GEBT. The parameters were tH=45.83 min and b=0.13 for the actual scintigraphy data, and tH=52.64 min and b=0.35 for the estimated scintigraphy data.

FIG. 4 shows a comparison of actual CGM glucose levels and levels predicted based on scintigraphy and GEBT. Actual scintigraphy values (blue diamonds), values predicted using scintigraphy data (red curve), and values predicted using GEBT (green curve). This shows that the CGM-glucose predicted from scintigraphy and the GEBT are both very similar to the actual CGM-glucose. The RMS difference between the two prediction curves was 4.68 mg/dl, and the AUC values were 21,749 and 21,148 respectively (a 2.8% difference). These data provide proof of concept that (i) GE can be estimated from GEBT values and (ii) GEBT can be used to predict accurately postprandial CGM values.

The inventors have performed studies to develop and validate the inventive aspects described herein. Aiming to quantify the relationship between GE and glycemia, these variables were simultaneously assessed once (n=5) or twice (n=10) with scintigraphy and 4 h continuous glucose monitoring (CGM) in 15 type 1 diabetes patients (A1c 8.7 [0.5]%) with normal (4 patients), delayed (9 patients), or rapid GE (2 patients). The actual and predicted mean 4 h postprandial glucose were compared; predicted glucose was derived by equations that incorporate the fasting glucose, caloric intake, insulin dose, and patient-specific or surrogate GE.

For the first study, the root-mean-square error between actual and predicted mean (SD) CGM glucose using patient-specific GE was 14.3 [6.4] mg/dl, but greater when predicted CGM glucose was derived from surrogate GE values (P<0.05). The simulations demonstrated that fasting glucose, GE, insulin sensitivity, and insulin administration time all affect postprandial glucose; when the short-acting insulin dose is administered 60 minutes after instead of 30 minutes before a meal, the predicted duration for which the 4 h postprandial blood glucose (4 h) is <70 mg/dl declined from 82 to 46%. In summary, it was found that GE is useful for accurately and precisely predicting and optimizing the post-prandial glucose profile in type 1 diabetes.

The upper gastrointestinal manifestations of diabetes include: (1) gastroparesis, which is characterized by severe upper gastrointestinal symptoms and markedly delayed GE. In the United States type 1 diabetes exchange clinic registry, 4.8% of adults who had diabetes for at least 2 years had a clinical diagnosis of gastroparesis; (2) dyspepsia, which is characterized by less severe symptoms and either normal, rapid, or modestly delayed GE; and (3) delayed GE with no or mild upper gastrointestinal symptoms. Indeed, greater than 40% of patients with sub-optimally controlled type 1 and 2 diabetes have delayed GE, which is generally associated with no or mild upper gastrointestinal symptoms. Because GE is not routinely evaluated in such patients, delayed GE may be under recognized.

Besides causing upper gastrointestinal symptoms, there is a bidirectional relationship between gastric emptying disturbances and postprandial glycemia. Hyperglycemia predisposes to delayed GE in type 1 diabetes. Conversely, delayed GE may affect control of glycemia. Indeed, the therapeutic benefits of GLP-1 agonists and pramlintide are at least partly explained by drug-induced decelerated GE. In addition, limited evidence suggests that delayed or rapid GE, perhaps secondary to gastrointestinal enteric or autonomic neuropathy, may perpetuate dysglycemia in diabetes. Among 49 patients with type 1 or type 2 diabetes, GE was more likely to be delayed in patients with (81%) than without (17%) hypoglycemia. Conceivably, when GE is delayed, insulin may act before nutrients are absorbed, predisposing to hypoglycemia. Conversely, rapid GE predisposes to postprandial hyperglycemia in type 1 diabetes.

These studies investigated the overall relationship between disturbances of gastric emptying and glycemia. By comparison, the precise temporal relationship between GE and postprandial glycemia is poorly understood. Intuitively, the rate of GE should influence the rate at which meal-related glucose enters the portal circulation. However, one study observed that excessive hepatic glucose release, not rapid entry of ingested glucose, was primarily responsible for postprandial hyperglycemia in type 2 diabetes. The current state-of-the-art mathematical models (e.g., oral minimal model), used to assess glucose metabolism in diabetes, assume the GE rate is the same in all patients. Studies that evaluated the diurnal patterns in insulin action and the impact of exercise on glucose in diabetes excluded participants with delayed GE. The consensus guidelines for diabetes do not recommend that GE be assessed, even in poorly controlled patients; increased glycemia or its variability is implicated to other factors such as poor compliance, suboptimal dosing or erratic absorption of insulin, and misestimated caloric intake. However, even AID systems improve but frequently fail to optimize glycemic control (i.e., A1c less than 7%) in diabetes. Further, closed loop systems, are by design, reactive rather than proactive, i.e., they increase or decrease insulin delivery to correct glycemic fluctuations, which may at least partly be due to GE disturbances.

Prompted by these gaps, the inventors performed studies that sought to understand better the temporal relationship between the GE rate and the postprandial glucose profile in type 1 diabetes patients who were receiving continuous subcutaneous insulin or multiple dose insulin injections. The aims were (1) to use the fasting glucose level, calories consumed, GE, and insulin administered to forecast the postprandial glucose profile and (2) through simulations, to explore the effects of adjusting the timing of insulin delivery on the postprandial glucose profile in type 1 diabetes with normal, delayed, or rapid GE. A better understanding of the relationship between GE and glycemia in type 1 diabetes is necessary because postprandial hyperglycemia is a major contributor not only to overall glycemic control (e.g., HbA1c) but also increases the risk of micro-vascular and macro-vascular complications in patients with diabetes. Conversely, more intensive insulin regimens are more likely to be associated with hypoglycemia, the pathophysiology of which is incompletely understood. The dose and timing of insulin delivery are critical to reducing glycemic variability in diabetes. It is conceivable that adjusting the dose and timing of insulin delivery based on GE will improve the control of glycemia in diabetes.

### Research Design and Methods

### Study Design and Participants

GE and continuous glucose monitoring (CGM) were simultaneously measured in 15 patients (8 women, mean [SD] age 53 years, BMI 28 [4] kg/m²) with type 1 diabetes for 5 years or longer (A1c 8.9 [1.2] %) who were receiving continuous subcutaneous insulin injections (CSII) (9 patients) or multiple daily injections (MDI) (6 patients). Patients on closed loop systems were not enrolled. All patients had 2 studies at an interval of seven days. The eligibility criteria did not include gastrointestinal symptoms. No patients were taking medications that affect gastrointestinal motility.

### Assessments

The participants recorded their gastrointestinal symptoms in the Gastroparesis Cardinal Symptom Index (Daily Diary) for one week after the GE study. GE of solids was assessed with a balanced solid meal (230 kcal, 18 g carbohydrates, 14.4 g protein, and 11.2 g fat) labeled with 0.5 mCi 99 mTc-sulfur colloid and 100 mg of C-Spirulina platensis. GE was simultaneously evaluated with scintigraphy and a CO₂ Breath Test; only the scintigraphy data are described here. GE t_{half} values less than 52 and greater than 86 minutes were considered to be rapid and delayed. The continuous glucose monitoring (CGM, DexCom G4 professional, DexCom) device was placed and calibrated with finger-stick glucose readings 2 hours before the GE study. During the 4 h GE study, subcutaneous glucose values were measured every 5 minutes with CGM device which was calibrated with glucose meter measured glucose concentration every 12 hours. Autonomic nervous system functions were assessed by standardized and validated techniques and compared to normative values. Postganglionic sympathetic sudomotor function was evaluated by the quantitative sudomotor axon reflex test and compared with normative data. Cardiovagal functions were evaluated with the heart rate response to deep breathing and Valsalva ratio. Cardiovascular adrenergic function was evaluated by measuring blood pressure and heart rate responses to Valsalva maneuver and head upright tilt. A semiquantitative composite autonomic severity score (CASS) ranging from 0 to 10 was calculated by combining sudomotor (range, 0-3), cardiovagal (range, 0-3), and adrenergic range, 0-4) scores adjusted for age and sex. Mild, moderate and severe autonomic neuropathy were defined by total score of 0-3, 4-6, 7-10 respectively.

### Data and Statistical Analysis

There were three key steps. First, the modified oral minimal model was adapted to forecast the postprandial CGM glucose profile for 4 hours from the following inputs: fasting (not postprandial) CGM glucose, calories consumed, insulin dose, and patent-specific GE values (Supplementary Methods). The same model was used for all analyses. Second, in each patient, the predicted CGM glucose profile was derived from the patient-specific GE values and separately from surrogate GE values. These surrogate GE values were derived by fitting the GE equations to the mean GE curve in patients who were known to have normal, delayed and rapid GE by scintigraphy. Finally, simulations (based on the model) were used to evaluate the effects of varying the time at which insulin was given relative to meals on the predicted CGM glucose profile. Similar to the GE scintigraphy meal used in this study, these simulations incorporated a carbohydrate intake of 18 gm at breakfast. Assuming a body weight of 60 kg and an insulin requirement of 0.6 U/kg, the estimated total daily insulin requirement was 36 U, of which 50% (i.e., 18 U) was 5 evenly distributed across 5 meals. Since breakfast comprised 20% of the daily caloric intake, we assumed that 3.6 units of rapid acting insulin was provided with the test meal at breakfast. The estimated total basal insulin dose (i.e., 18 U) was administered in 2 daily doses of glargine (9 U) at an interval of 12 h. These simulations incorporated representative values for GE (i.e., observed median values for GE t_{half} and beta in participants with normal, rapid, and delayed GE), fasting blood glucose (i.e., 10 mg/dl increments between 70 and 200 mg/dl), time of administration of prandial insulin (i.e., 10 minute intervals between 30 minutes before to 1 h after the meal), and insulin sensitivity (i.e., lowest, middle, and greatest published values for insulin sensitivity in health and diabetes).

The goodness of fit between the actual and predicted average postprandial 4 hr CGM glucose values, which were derived from the patient-specific and surrogate (normal, delayed, and rapid) GE values, was evaluated with the root-mean-square [RMS] error. Lin's Concordance Correlation Coefficient (CCC) evaluated the relationship between the GE t half for first and second studies.

The models that evaluated the effects of varying the time of insulin administration on the 4 h mean postprandial glucose level considered nine combinations of insulin sensitivity (i.e., low, moderate or high) and GE (i.e., normal, low, or rapid). The equations were used to predict the 4 h mean postprandial glucose level for each of these nine combinations, which were analysed by separate linear regression models. These models evaluated the extent to which the predictor variables (i.e., fasting glucose level, the time of prandial insulin administration, and their interaction), when used as linear continuous variables, explained the predicted 4 h mean postprandial glucose level. All continuous variables are expressed as mean (SD). A P value less than 0.05 was considered significant. The mathematical modeling to predict post prandial glycemia was done in MATLAB (MathWorks Inc., Natick, MA). Statistical analysis was performed using JMP Pro 14 (SAS Institute) and R-3.6.3.

### Results

### Clinical Features

On average, patients had poorly controlled type 1 diabetes with a duration of diabetes of 30 years. The demographic features and complications of diabetes were not significantly different among patients with normal, delayed or rapid GE (see Table 1, below). Twelve patients (80%) had one or more longer-term complications of diabetes, i.e., a retinopathy (12 patients), neuropathy (5 patients) or nephropathy (6 patients). Ten of 15 patients (67%) had autonomic neuropathy, with 7 (70%) patients having mild autonomic neuropathy. The gastrointestinal symptom scores suggest that on average, patients had very mild or mild symptoms.

TABLE 1

| | | | |
|---|---|---|---|
| | Rapid GE | Normal GE | Delayed GE |
| Parameters | (n = 2) | (n = 4) | (n = 9) |

Demographic features

| | | | |
|---|---|---|---|
| Age, y | 50 (17) | 56 (19) | 54 (17) |
| BMI, kg/m² | 29 (2) | 26 (6) | 28 (3) |

Diabetes mellitus

| | | | |
|---|---|---|---|
| Duration of diabetes, y | 30 (35) | 35 (24) | 26 (12) |
| HbA1C, % | 8.7 (0.1) | 8.9 (0.5) | 8.9 (0.6) |
| Fasting blood glucose, mg/dl¹ | 123 (40) | 152 (41) | 131 (50) |
| Continuous subcutaneous insulin | 2, 0 | 3, 1 | 4, 5 |
| infusion (CSII), multiple dose | | | |
| insulin (MDI), n | | | |

Complications

| | | | |
|---|---|---|---|
| Neuropathy (CSII, MDI), n | 0, — | 1, 0 | 2, 2 |
| Retinopathy (CSII, MDI), n | 1, — | 3, 1 | 2, 5 |
| Nephropathy (CSII, MDI), n | 0, — | 3, 0 | 1, 2 |
| Autonomic Neuropathy: | 1, 0, 0 | 2, 1, 0 | 4, 1, 1 |
| mild, moderate, severe, n | | | |

Gastrointestinal symptoms

| | | | |
|---|---|---|---|
| GCSI-DD Total score^{†C} | 0 (0, 0) | 0.19 (0, 2.6) | 0.9 (0.7, 2.1) |
| Post-prandial fullness*^{†C} | 0 (0, 0) | 0.2 (0, 1.6) | 0.3 (0.2, 1.3) |
| Early satiety^{†C} | 0 (0, 0) | 0 (0, 1.5) | 0.4 (0.3, 1) |
| Nausea^{†C} | 0 (0, 0) | 0 (0, 1.3) | 0 (0, 0.3) |
| Bloating^{†C} | 0 (0, 0) | 0 (0, 1) | 0.5 (0.3, 0.6) |
| Upper abdominal paint^{†C} | 0 (0, 0) | 0 (0, 0.6) | 0.4 (0, 1) |

Postprandial glycemia

| | | | |
|---|---|---|---|
| PPG increment¹ (mg/dl) | 56 (9) | 66 (48) | 37 (16) |
| $\begin{matrix}Insulin {sensitivity}^{1} \times \\ {10}^{- 4} ( \frac{{min}^{- 2} , mL}{μ U} )\end{matrix}$ | 6 (2) | 2 (0.8) | 7 (5) |
| RMS Error between actual and | 10 (6) | 14 (3) | 14 (9) |
| predicted CGM (mg/dl)¹ | | | |

GCSI-DD - Gastroparesis Cardinal Symptom Index - Daily Diary
All data are Mean (SD), except ^{†} which is Median (IQR)
¹Before first GE study in patients who had 2 studies
*P <0.05, by Kruskal Wallis test, followed by pairwise Wilcoxon rank-sum test
^{C}: Symptoms and QOL are ranked on a scale of 0 (none) to S (very severe), where 1 represents very mild symptoms,

### Reproducibility

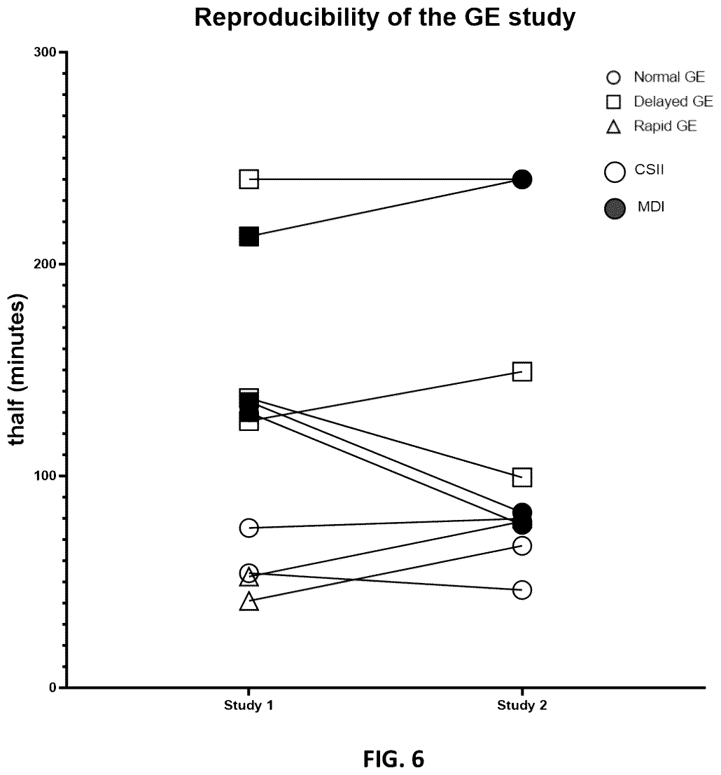

5 Five of 30 studies were not analysed because patients received dextrose to treat hypoglycaemia (n=2) or because the CGM recording (n=2) or scintigraphy were suboptimal (n=1). Hence, one and 2 studies were analysed in 5 and 10 patients. Among these 10 patients, the GE t_{half} was 160 (56) and 202 (85) minutes for the first and second studies (FIG. 6); the concordance correlation coefficient (CCC) for these GE studies was 0.47 (95% CI 0.09-0.80). Of 15 patients, 4 (27%), nine (60%), and two patients (13%) had normal, delayed, and rapid GE in the first study (Table 1). The insulin dose was different between the first and second studies in six patients. In five patients, the GE was normal (one patient) or delayed (four patients) in both studies. In the remaining five patients, i.e., the GE was normal vs rapid (three patients) or normal vs delayed (two patients) (FIG. 6).

### Actual Vs Predicted CGM Glucose

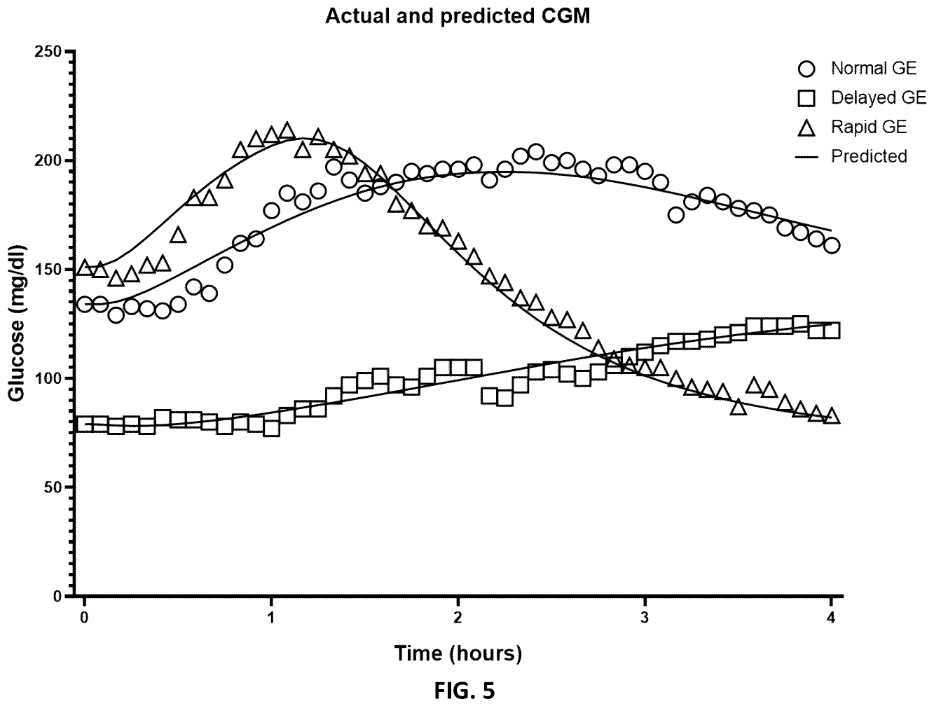

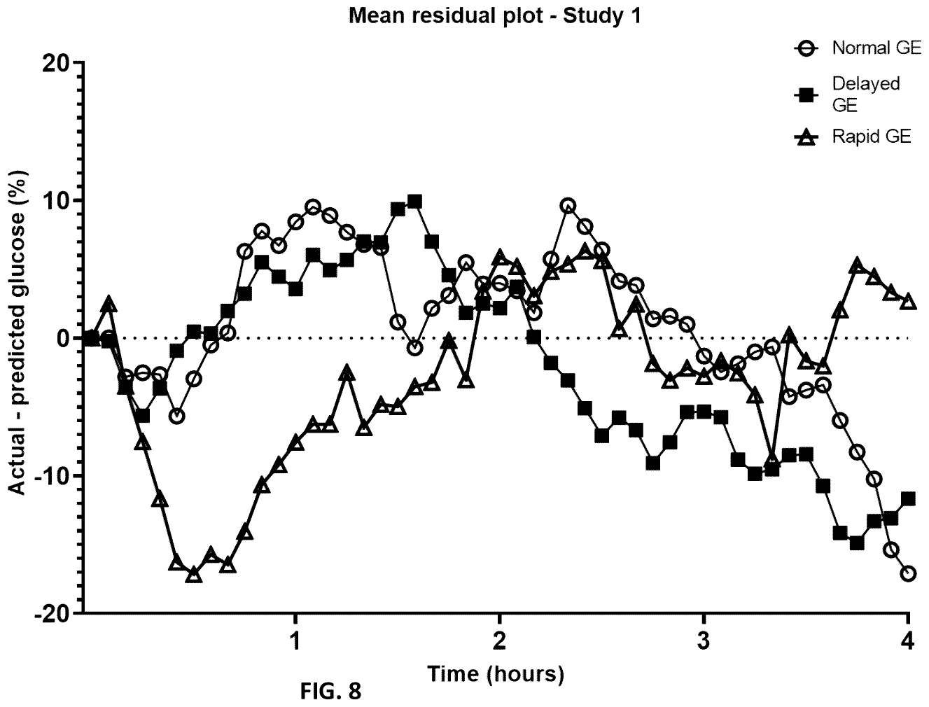

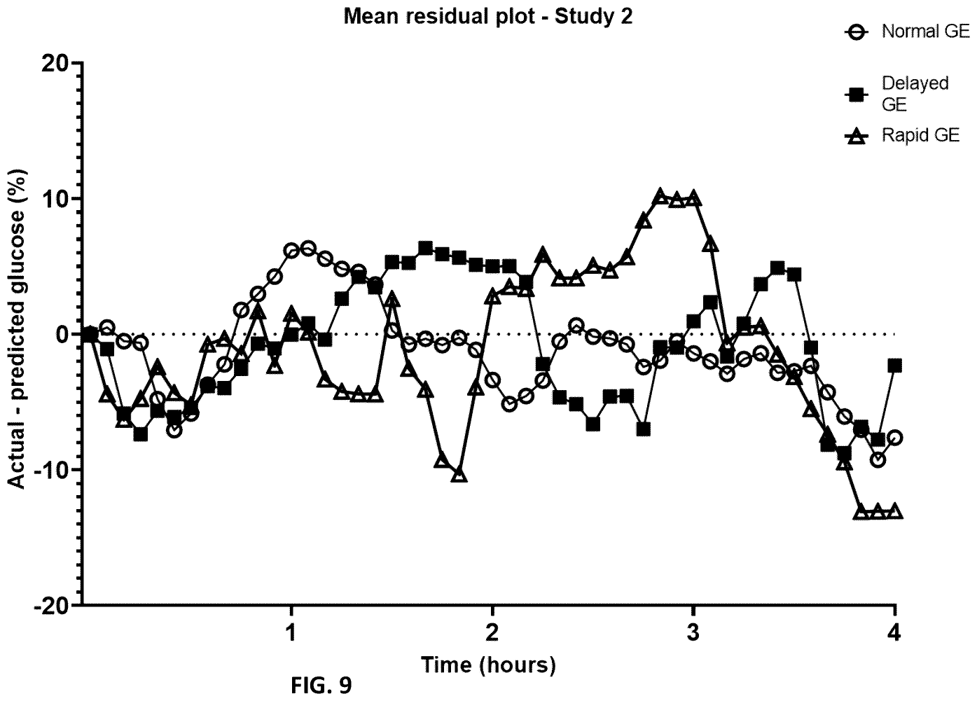

In most patients, the predicted CGM glucose profile closely approximated the actual CGM glucose profile (FIG. 5). Across all participants, the average difference between the actual and predicted CGM glucose value at each time ranged from 0 to 20% (FIGS. 8 and 9). For the first study, the RMS error for the difference between the predicted CGM glucose level derived from the patient-specific GE value and the actual CGM glucose level was 14.3 [6.4] mg/dl (FIG. 8).

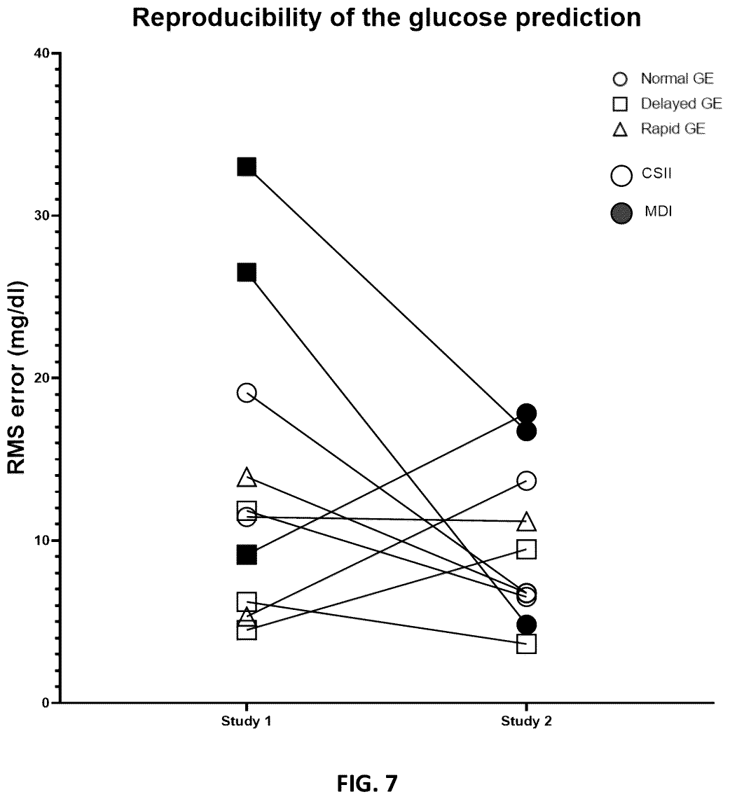

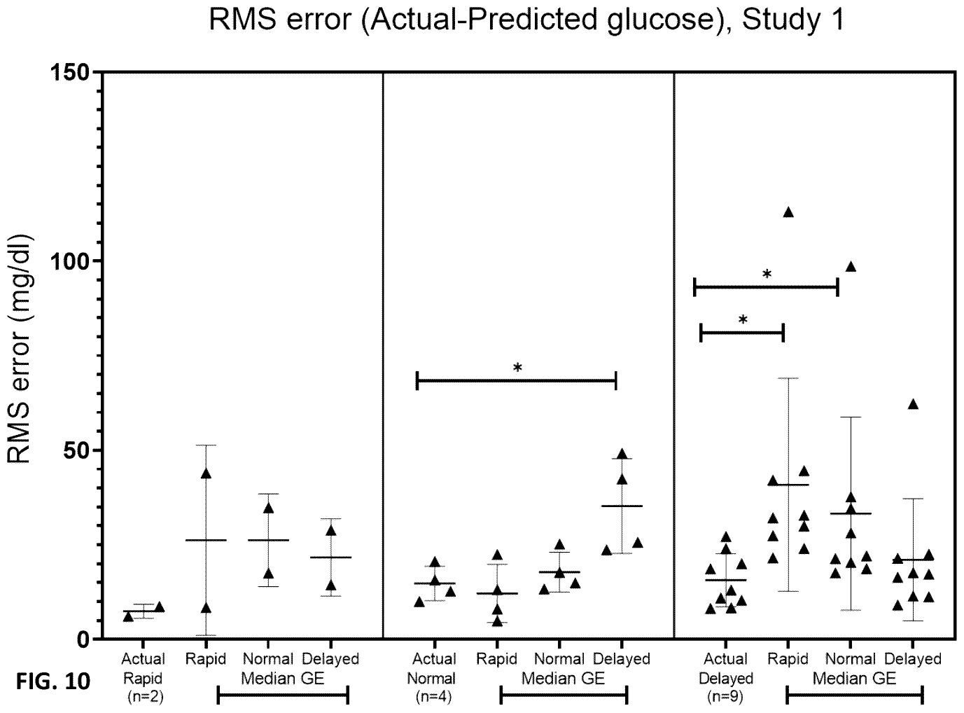

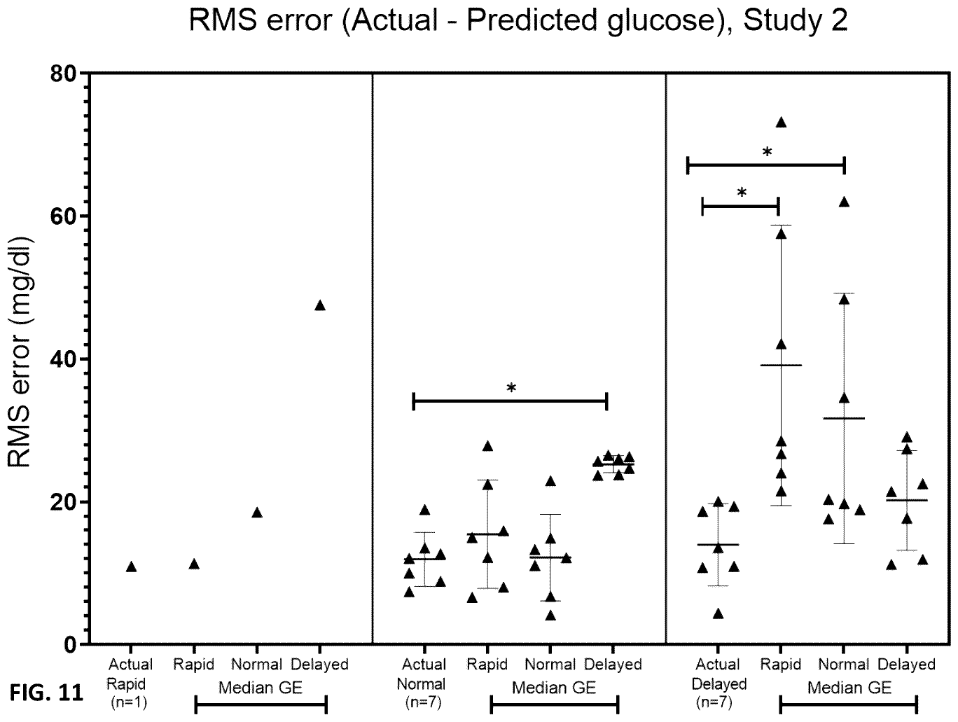

The predicted CGM glucose was also derived by substituting the patient-specific GE data with surrogate GE values. These surrogate GE values were the median values in patients with normal, delayed, and rapid GE. Among patients with normal GE, the RMS error between actual and predicted CGM profiles was greater when surrogate-delayed GE rather than the patient-specific GE data were used to derive the predicted CGM profile (P=0.03). Likewise, among patients with delayed GE, the RMS error was greater when the predicted CGM profile was derived from surrogate-normal GE (P=0.01) or surrogate-rapid GE rather than patient-specific GE value (P=0.001) (FIG. 10). Similar differences between actual and predicted CGM profiles derived from patient specific and surrogate GE values were observed in the second study (FIG. 11). Despite differences in the insulin dose and GE between the first and second studies, the RMS errors between actual and predicted average 4 h postprandial CGM glucose were within 15-30 mg/dl in 8 of 10 patients, which corresponds to a change of 0.5-1% in the HbA1c over a period of time (FIG. 7). Effects of Adjusting the Time of Prandial Insulin Administration on

### Glycemia

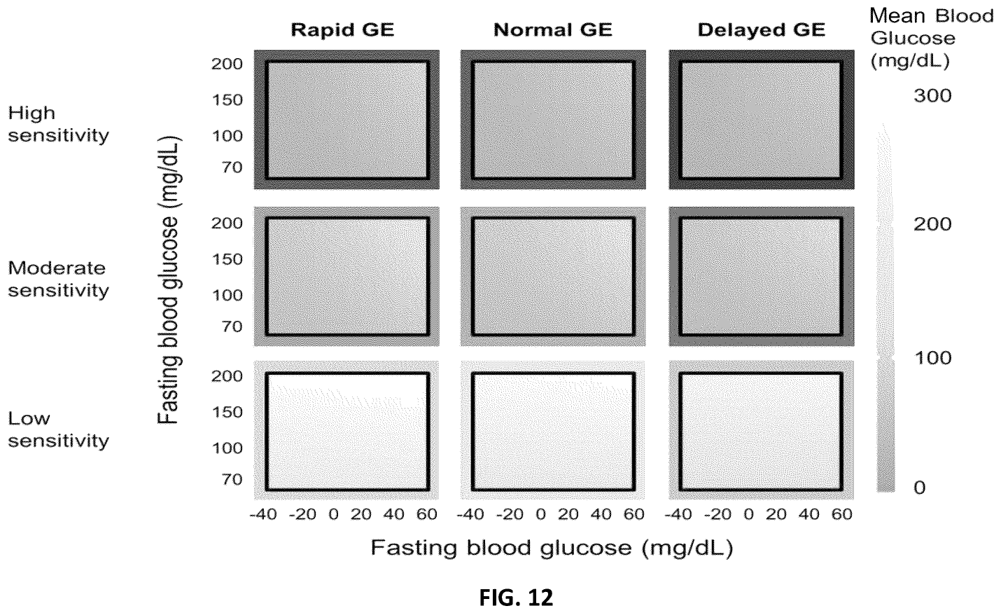

The simulations explored the effects of adjusting the time of prandial insulin administration on the mean postprandial 4 hr CGM glucose profile (FIG. 12). These simulations were individualized by insulin sensitivity (i.e., low, moderate, and high) and GE (i.e., normal, rapid, and delayed). Hence, there are nine combinations, each of which explores the effects of adjusting the insulin administration on the 4 h CGM glucose across a range of values for fasting blood glucose and insulin administration times.

The average 4 h postprandial CGM glucose level was associated with insulin sensitivity (P<0.001, Wilcoxon rank sum test). The 4 h postprandial CGM glucose was greater in patients with low than moderate and greater in those with moderate than high insulin sensitivity. The average 4 h postprandial CGM glucose level was also associated with GE (P<0.001, Wilcoxon rank sum test). The average 4 h CGM glucose level was greater in patients with rapid vs normal and greater in normal vs delayed GE (P<0.001).

Nine separate linear regression models, which correspond to the nine panels in FIG. 12, analysed the effects of adjusting the timing of the prandial insulin dose relative to meals, and of the fasting blood glucose, on the average 4 h CGM glucose. In all nine models, the fasting blood glucose, insulin administration time, and the interaction term (fasting blood glucose insulin administration time) predicted the 4 hr postprandial CGM glucose (P<0.0001). For all these terms, the parameter estimates were positive, which suggests that when the fasting blood glucose was greater, and when the interval between the meal and the postprandial insulin dose was greater, the 4 hr postprandial CGM glucose was greater. The positive interaction suggests that there was a greater effect of an increase in each variable when the other variable was greater (synergy). Conversely, when the insulin was administered before the meal, the 4 hr postprandial CGM glucose was lower.

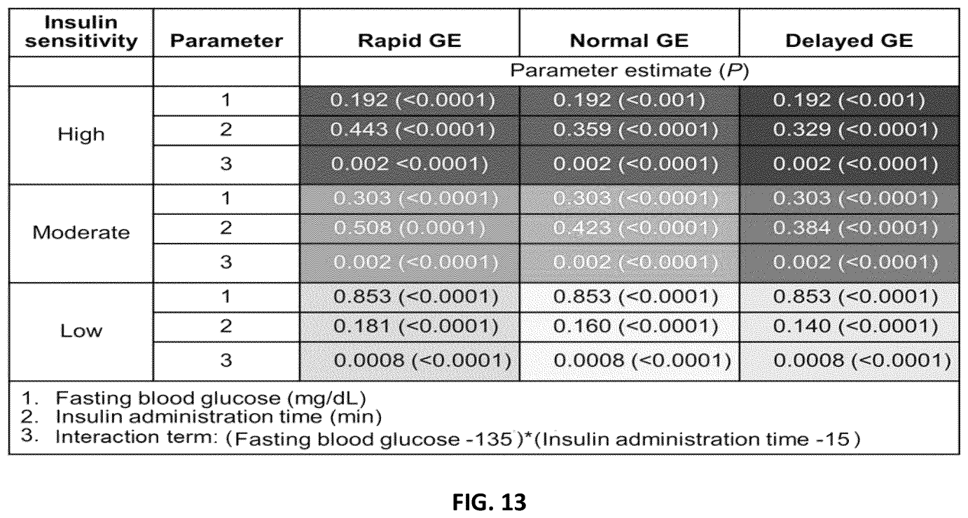

The parameter estimates for these terms varied among the models (FIG. 13). For fasting blood glucose, the parameter estimates were approximately 0.19, 0.30, and 0.85 (P<0.0001) in patients with high, medium, and low insulin sensitivity, regardless of the GE. This suggests that on average, for every 1 mg/dl positive increment in fasting blood glucose, the average 4 h postprandial CGM increases by approximately 0.19, 0.30, and 0.85 mg/dl in patients with high, medium, and low insulin sensitivity groups, regardless of GE. However, within each insulin sensitivity category (i.e., low, moderate and high), the parameter estimates for timing of insulin administration were influenced by GE. For example, in patients with low insulin sensitivity (e.g., suggestive of type 1 diabetes), the parameter estimates for timing were approximately 0.14, 0.16, and 0.18 (P<0.0001) in patients with delayed, normal, and rapid GE. This suggests that when insulin is given before a meal, every incremental 1-minute advancement in the timing is associated with a reduction in the average 4 h postprandial CGM by 0.14, 0.16, and 0.18 mg/dl in patients with delayed, normal, and rapid GE. Conversely, when insulin is administered after a meal, every minute's delay is associated with an increase in the average 4 h postprandial CGM by these same amounts in patients with delayed, normal, and rapid GE. The interaction terms (i.e., fasting blood glucose insulin administration time) signify that the effects of insulin administration time on the predicted average 4 h postprandial CGM are more pronounced when the fasting blood glucose is greater.

Table 2 (below) shows the predicted effects of adjusting the insulin administration time on postprandial blood glucose (4 h) in a type 1 diabetes patient with moderate insulin sensitivity, fasting blood glucose of 130 mg/dl, and delayed GE. When the short-acting insulin dose is administered 60 minutes after instead of 30 minutes before a meal, the duration for which the 4 h postprandial blood glucose (4 h) is <70 mg/dl declines from 82 to 46%.

TABLE 2

| | Mean | | | |
|---|---|---|---|---|
| Time of administration | blood | % | % time | % |
| of prandial insulin | glucose | time <70 | 70-180 | time >180 |
| (minutes) (T = mealtime) | (mg/dl) | mg/dl | mg/dl | mg/dl |
| T − 30 | 49 | 82 | 18 | 0 |
| T − 20 | 50 | 79 | 21 | 0 |
| T − 10 | 53 | 75 | 25 | 0 |
| T − 0 (with meal) | 56 | 71 | 29 | 0 |
| T + 10 | 60 | 66 | 34 | 0 |
| T + 20 | 64 | 62 | 38 | 0 |
| T + 30 | 68 | 58 | 42 | 0 |
| T + 40 | 72 | 54 | 46 | 0 |
| T + 50 | 77 | 50 | 50 | 0 |
| T + 60 | 81 | 46 | 54 | 0 |

### DISCUSSION

The classical triple-tracer studies of glucose metabolism deconvolve the measured meal-related glucose appearance to estimate the rates of GE and intestinal glucose absorption. However, these equations are not based on experiments in which GE was measured. By contrast, GE and CGM were simultaneously measured in this study. The equations, which were adapted from the oral minimal model, were used to predict the postprandial CGM glucose from the ingested carbohydrate load, the GE rate, and the insulin dose. By contrast to the oral minimal model, our model does not include plasma insulin or C-peptide levels because endogenous insulin production is minimal or non-existent in type 1 diabetes. The findings suggest that the predicted CGM glucose closely approximated the actual postprandial CGM glucose profile over 4 hours in type 1 diabetes patients with normal, delayed, or rapid GE who were receiving continuous subcutaneous or multiple daily insulin injections. In most participants, the difference between the actual and the predicted CGM was less than the clinically acceptable range of 20%, even though the glucose profile, insulin regimens, and GE rate varied among and within patients. Indeed, the GE varied considerably between days in 5 of 10 patients (50%). This day-to-day variability in GE and the CGM profile did not affect the accuracy of the model-predicted CGM glucose. Underscoring the contribution of the GE rate to the postprandial glucose, the error between the actual and predicted CGM glucose was greater when the patient-specific GE value was replaced by a surrogate value.

These patients were representative of the poorly controlled patients with type 1 diabetes seen by endocrinologists. Of the patients, 80% had one or more complications and, confirming earlier studies, 60% of patients had delayed GE, albeit with no or mild GI symptoms. The ability to predict postprandial hyperglycemia is clinically relevant because postprandial hyperglycemia accounts for ˜60% of total hyperglycemia in type 1 diabetes and accounts for most of the hyperglycemia that pose a challenge to closed loop systems. The simulations suggest that the individualized timing of insulin meal bolus delivery based on the fasting CGM glucose, GE, and insulin sensitivity may improve control of glycemia. These simulations suggest that hypoglycemia and hyperglycemia are more likely in patients with delayed and rapid GE respectively. Moreover, the models predict that administering insulin after instead of before a meal may reduce the risk of a blood glucose level that is less than 70 mg/dl in patients with type 1 diabetes, delayed GE, moderate insulin sensitivity, and a fasting blood glucose of 130 mg/dl. Conversely, delayed administration of insulin may predispose to a higher postprandial glucose level in patients with rapid GE. Indeed, the rate of increase in postprandial glucose was respectively slower and faster when GE was delayed and accelerated in patients with type 1 diabetes. The findings of these simulations need to be confirmed by studies.

Our study has certain limitations. Only two patients had rapid GE, which is uncommon in type 1 diabetes. The model, which was adapted from the oral minimal model, assumes that postprandial glucose is derived entirely from ingested glucose, and, in contrast to some other models, ignores the relatively small contribution of endogenous glucose production to postprandial glucose. The estimated insulin pharmacokinetic parameters and insulin sensitivity used in this model were not measured but rather based on published values.

In summary, these studies confirmed that the post-prandial glucose profile can be accurately and precisely predicted from the GE rate in patients with type 1 diabetes. These findings support consideration of assessing GE in patients with suboptimal glycemic control, even in patients with no or mild gastrointestinal symptoms. They also provide the basis for mechanistic and therapeutic studies that evaluate the relationship between GE and glycemia in diabetes.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method of controlling blood glucose levels of a patient, the method comprising delivering one or more dosage amounts of insulin to the patient at a particular time relative to a meal, wherein the one or more dosage amounts and a timing of delivery are determined based on a combination of factors specific to the patient that include: (i) a fasting glucose level, (ii) ingested calories, and (iii) a gastric emptying value.

2. The method of claim 1, comprising:
determining the gastric emptying value of the patient using Gastric Emptying Breath Test; and
delivering one or more dosage amounts of insulin to the patient based on the determined Gastric Emptying Breath Test.

3. The method of claim 1, comprising:
determining the gastric emptying value of the patient using scintigraphic data; and
delivering one or more dosage amounts of insulin to the patient based on the gastric emptying value.

4. The method of claim 1, wherein the gastric emptying value is categorized as rapid, normal, or delayed.

5. The method of claim 1, comprising:
determining the fasting glucose level of the patient by using continuous glucose monitoring; and
adjusting one or more dosage amounts of insulin to the patient based on the fasting glucose level determined by continuous glucose monitoring.

6. The method of claim 1, further comprising:
adjusting a timing of delivering the one or more dosage amounts of insulin to the patient, wherein the patient's gastric emptying value is used determining the adjusted timing.

7. The method of claim 1, further comprising:
evaluating the patient's insulin sensitivity;
determining the one or more dosage amounts of insulin to the patient based on the evaluated patient's insulin sensitivity, the fasting glucose level of the patient, the ingested calories of the patient, and the gastric emptying value of the patient.

8. The method of claim 1, further comprising validating an accuracy of the gastric emptying value by comparing predicted glucose levels to actual glucose levels over a predetermined time period.

9. The method of claim 1, wherein the patient is identified as having Type-1 diabetes and exhibit either rapid or delayed gastric emptying.

10. A method of treating a patient having Type-1 diabetes, the method comprising:
determining one or more dosage amounts of insulin based on a combination of factors specific to the patient that include: (i) a fasting glucose level, (ii) ingested calories, and (iii) a gastric emptying value; and
delivering one or more dosage amounts of insulin to the patient.

11. The method of claim 10, further comprising administering the one or more dosage amounts of insulin time intervals that are relative to a meal consumption, wherein the time intervals are from about 30 minutes before to about 60 minutes after the meal.

12. The method of claim 10, wherein the one or more dosage amounts of insulin are delivered through continuous subcutaneous insulin injections (CSII) or multiple daily injections (MDI).

13. The method of claim 10, further comprising assessing autonomic nervous system functions through standardized tests comprising one or more of quantitative sudomotor axon reflex test, heart rate response to deep breathing, Valsalva ratio, and responses to a head upright tilt.

14. The method of claim 13, wherein the assessment of autonomic nervous system functions comprises calculating a composite autonomic severity score to define an extent of autonomic neuropathy, and the one or more dosage amounts of insulin are further determined based on the composite autonomic severity score.
